(19) United States
(12) Patent Application Publication
(10) Pub. No.: US 2026/0251891 A1
(43) Pub. Date: Aug. 27, 2026

(54) APPARATUS FOR REDUCTION OF SIGNAL VARIATION IN SEQUENCING SYSTEM

(71) Applicant: ILLUMINA, INC., San Diego, CA (US)

(72) Inventors: Steven BOEGE, San Mateo, CA (US); Changqin DING, Foster City, CA (US); Danilo CONDELLO, Madrid (ES)

(21) Appl. No.: 18/877,291

(22) PCT Filed: Jun. 27, 2023
(86) PCT No.: PCT/US2023/026275

§ 371 Date: Dec. 20, 2024

Related U.S. Application Data
(60) Provisional application No. 63/357,074, filed on Jun. 30, 2022.

(51) Int. Cl. G02B 21/06; G02B 21/26

(52) U.S. Cl. CPC G02B 21/06; G02B 21/26

(57) ABSTRACT

An apparatus includes a sequencing stage and an illumination assembly. The sequencing stage is configured to receive a flow cell that includes a. plurality of reaction sites. Each reaction site is configured to contain a biological sample. The illumination assembly is configured to project light toward the sequencing stage to thereby illuminate the reaction sites. The illumination assembly includes a light source, a first lenslet array, a second lenslet array, and a diffuser. The first lenslet array is interposed between the light source and the sequencing stage. The second lenslet array is also interposed between the light source and the sequencing stage. The diffuser is also interposed between the light source and the sequencing stage.

### PRIORITY

This application claims priority to U.S. Provisional Pat. App. No. 63/357,074, entitled “Apparatus for Reduction of Signal Variation in Sequencing System,” filed Jun. 30, 2022, the disclosure of which is incorporated by reference herein, in its entirety.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves may also correspond to implementations of the claimed technology.

Aspects of the present disclosure relate generally to biological or chemical analysis and more particularly to systems and methods using image sensors for biological or chemical analysis.

Various protocols in biological or chemical research involve performing a large number of controlled reactions on local support surfaces or within predefined reaction chambers. The designated reactions may then be observed or detected, and subsequent analysis may help identify or reveal properties of chemicals involved in the reaction. For example, in some multiplex assays, an unknown analyte having an identifiable label (e.g., fluorescent label) may be exposed to thousands of known probes under controlled conditions. Each known probe may be deposited into a corresponding well of a flow cell channel. Observing any chemical reactions that occur between the known probes and the unknown analyte within the wells may help identify or reveal properties of the analyte. Other examples of such protocols include known DNA sequencing processes, such as sequencing-by-synthesis (SBS) or cyclic-array sequencing.

In some conventional fluorescent-detection protocols, an optical system is used to direct an excitation light onto fluorescently-labeled analytes and to also detect the fluorescent signals that may be emitted from the analytes. Such optical systems may include an arrangement of lenses, filters, and light sources. It may be desirable to provide uniformity in spatial irradiance from the excitation light, to thereby obtain spatial uniformity in the fluorescence emission signal collected. However, providing such uniformity in spatial irradiance from the excitation light, to thereby obtain spatial uniformity in the fluorescence emission signal collected, may present challenges.

### BRIEF DESCRIPTION OF THE DRAWINGS

FIG. 1 depicts a schematic diagram of an example of an imaging system that may be implemented in a system for biological or chemical analysis.

FIG. 2 depicts a perspective view of an example of an optical fiber having a core with a square cross-sectional profile.

FIG. 3 depicts a graph plotting examples of modulation magnitudes as a function of frequency, as obtained using the optical fiber of FIG. 2 in two different wavelength ranges.

FIG. 4 depicts a perspective view of an example of an optical fiber having a core with a circular cross-sectional profile.

FIG. 5 depicts a graph plotting examples of modulation magnitudes as a function of frequency, as obtained using the optical fiber of FIG. 4 in two different wavelength ranges.

FIG. 6 depicts a perspective view of an example of an optical fiber having a core with a rectangular cross-sectional profile.

FIG. 7 depicts a graph plotting examples of modulation magnitudes as a function of frequency, as obtained using the optical fiber of FIG. 6 in two different wavelength ranges.

FIG. 8 depicts a schematic view of an example of an illuminating assembly.

FIG. 9 depicts a plan view of an illumination footprint of the illuminating assembly of FIG. 8 at a first plane.

FIG. 10 depicts a plan view of an illumination footprint of the illuminating assembly of FIG. 8 at a second plane.

FIG. 11 depicts a schematic view of another example of an illuminating assembly.

FIG. 12 depicts a schematic view of another example of an illuminating assembly.

FIG. 13 depicts a graph plotting examples of modulation magnitude as a function of frequency, as obtained using the optical fiber of FIG. 2 in the illuminating assembly of FIG. 12.

FIG. 14 depicts a graph plotting examples of modulation magnitude as a function of frequency, as obtained using the optical fiber of FIG. 4 in the illuminating assembly of FIG. 12.

FIG. 15 depicts a graph plotting examples of modulation magnitude as a function of frequency, as obtained using the optical fiber of FIG. 6 in the illuminating assembly of FIG. 12.

FIG. 16 depicts a schematic view of another example of an illuminating assembly.

FIG. 17 depicts a schematic view of another example of an illuminating assembly.

### DETAILED DESCRIPTION

### I. Overview of System for Biological or Chemical Analysis

Described herein are devices, systems, and methods for providing uniformity in spatial irradiance from excitation light emitted in an optical system, to thereby obtain spatial uniformity in fluorescent signals emitted from analytes that are exposed to the excitation light. Examples described herein may be used in various biological or chemical processes and systems for academic analysis, commercial analysis, or other analysis. More specifically, examples described herein may be used in various processes and systems where it is desired to detect an event, property, quality, or characteristic that is indicative of a designated reaction.

Bioassay systems such as those described herein may be configured to perform a plurality of designated reactions that may be detected individually or collectively. The biosensors and bioassay systems may be configured to perform numerous cycles in which a plurality of designated reactions occurs in parallel. For example, the bioassay systems may be used to sequence a dense array of DNA features through iterative cycles of enzymatic manipulation and image acquisition. Cartridges and biosensors that are used in the bioassay systems may include one or more microfluidic channels that deliver reagents or other reaction components to a reaction site. The reaction sites may be randomly distributed across a substantially planar surface; or may be patterned across a substantially planar surface in a predetermined manner. In some versions, the reaction sites are located in reaction chambers that compartmentalize the designated reactions therein.

Regardless of the form taken by the reaction sites, each of the reaction sites may be imaged to detect light from the reaction site. In some examples, one or more image sensors may detect light emitted from reaction sites. The signals indicating photons emitted from the reaction sites and detected by the individual image sensors may be referred to as those sensors' illumination values. These illumination values may be combined into an image indicating photons as detected from the reaction sites. These images may be further analyzed to identify compositions, reactions, conditions, etc., at each reaction site.

The following detailed description of certain examples will be better understood when read in conjunction with the appended drawings. To the extent that the figures illustrate diagrams of the functional blocks of various examples, the functional blocks are not necessarily indicative of the division between hardware components. Thus, for example, one or more of the functional blocks (e.g., processors or memories) may be implemented in a single piece of hardware (e.g., a general purpose signal processor or random access memory, hard disk, or the like). Similarly, the programs may be stand-alone programs, may be incorporated as subroutines in an operating system, may be functions in an installed software package, and the like. It should be understood that the various examples are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one example” are not intended to be interpreted as excluding the existence of additional examples that also incorporate the recited features. Moreover, unless explicitly stated to the contrary, examples “comprising” or “having” an element or a plurality of elements having a particular property may include additional elements whether or not they have that property.

As used herein, a “designated reaction” includes a change in at least one of a chemical, electrical, physical, or optical property (or quality) of an analyte-of-interest. In some examples, the designated reaction is a positive binding event (e.g., incorporation of a fluorescently labeled biomolecule with the analyte-of-interest). More generally, the designated reaction may be a chemical transformation, chemical change, or chemical interaction. In some examples, the designated reaction includes the incorporation of a fluorescently-labeled molecule to an analyte. The analyte may be an oligonucleotide and the fluorescently-labeled molecule may be a nucleotide. The designated reaction may be detected when an excitation light is directed toward the oligonucleotide having the labeled nucleotide, and the fluorophore emits a detectable fluorescent signal. In alternative examples, the detected fluorescence is a result of chemiluminescence or bioluminescence. A designated reaction may also increase fluorescence (or Förster) resonance energy transfer (FRET), for example, by bringing a donor fluorophore in proximity to an acceptor fluorophore, decrease FRET by separating donor and acceptor fluorophores, increase fluorescence by separating a quencher from a fluorophore or decrease fluorescence by co-locating a quencher and fluorophore.

As used herein, a “reaction component” or “reactant” includes any substance that may be used to obtain a designated reaction. For example, reaction components include reagents, enzymes, samples, other biomolecules, and buffer solutions. The reaction components may be delivered to a reaction site in a solution and/or immobilized at a reaction site. The reaction components may interact directly or indirectly with another substance, such as the analyte-of-interest.

As used herein, the term “reaction site” is a localized region where a designated reaction may occur. A reaction site may include support surfaces of a substrate where a substance may be immobilized thereon. For example, a reaction site may include a substantially planar surface in a channel of a flow cell that has a colony of nucleic acids thereon. The nucleic acids in the colony may have the same sequence, being for example, clonal copies of a single stranded or double stranded template. However, in some examples a reaction site may contain only a single nucleic acid molecule, for example, in a single stranded or double stranded form. Furthermore, a plurality of reaction sites may be randomly distributed along the support surface or arranged in a predetermined manner (e.g., side-by-side in a matrix, such as in microarrays). A reaction site may also include a reaction chamber that at least partially defines a spatial region or volume configured to compartmentalize the designated reaction. As used herein, the term “reaction chamber” includes a spatial region that is in fluid communication with a flow channel. The reaction chamber may be at least partially separated from the surrounding environment or other spatial regions. For example, a plurality of reaction chambers may be separated from each other by shared walls. As a more specific example, the reaction chamber may include a cavity defined by interior surfaces of a well and have an opening or aperture so that the cavity may be in fluid communication with a flow channel. Reaction sites do not necessarily need to be provided in reaction chambers and may instead be provided on or in any other suitable kind of structure.

As used herein, the term “adjacent” when used with respect to two reaction sites means no other reaction site is located between the two reaction sites. The term “adjacent” may have a similar meaning when used with respect to adjacent detection paths and adjacent image sensors (e.g., adjacent image sensors have no other image sensor therebetween). In some cases, a reaction site may not be adjacent to another reaction site; but may still be within an immediate vicinity of the other reaction site. A first reaction site may be in the immediate vicinity of a second reaction site when fluorescent emission signals from the first reaction site are detected by the image sensor associated with the second reaction site. More specifically, a first reaction site may be in the immediate vicinity of a second reaction site when the image sensor associated with the second reaction site detects, for example, crosstalk from the first reaction site. Adjacent reaction sites may be contiguous such that they abut each other or the adjacent sites may be non-contiguous having an intervening space between.

As used herein, a “substance” includes items or solids, such as capture beads, as well as biological or chemical substances. As used herein, a “biological or chemical substance” includes biomolecules, samples-of-interest, analytes-of-interest, and other chemical compound(s). A biological or chemical substance may be used to detect, identify, or analyze other chemical compound(s), or function as intermediaries to study or analyze other chemical compound(s). In particular examples, the biological or chemical substances include a biomolecule. As used herein, a “biomolecule” includes at least one of a biopolymer, nucleoside, nucleic acid, polynucleotide, oligonucleotide, protein, enzyme, polypeptide, antibody, antigen, ligand, receptor, polysaccharide, carbohydrate, polyphosphate, cell, tissue, organism, or fragment thereof or any other biologically active chemical compound(s) such as analogs or mimetics of the aforementioned species.

Biomolecules, samples, and biological or chemical substances may be naturally occurring or synthetic and may be suspended in a solution or mixture within a spatial region. Biomolecules, samples, and biological or chemical substances may also be bound to a solid phase or gel material. Biomolecules, samples, and biological or chemical substances may also include a pharmaceutical composition. In some cases, biomolecules, samples, and biological or chemical substances of interest may be referred to as targets, probes, or analytes.

As used herein, when the terms “removably” and “coupled” (or “engaged”) are used together to describe a relationship between components, the term is intended to mean that a connection between the components is readily separable without destroying or damaging the components. Components are readily separable when the components may be separated from each other without undue effort, or without a significant amount of time spent, in separating the components. For example, components may be removably coupled or engaged in an electrical manner such that the mating contacts of the components are not destroyed or damaged. Components may also be removably coupled or engaged in a mechanical manner such that the features that hold a component are not destroyed or damaged. Components may also be removably coupled or engaged in a fluidic manner such that ports of a component are not destroyed or damaged. The component is not considered to be destroyed or damaged if, for example, only a simple adjustment to the component (e.g., realignment) or a simple replacement (e.g., replacing a nozzle) is required.

As used herein, the term “fluid communication” or “fluidically coupled” refers to two spatial regions being connected together such that a liquid or gas may flow between the two spatial regions. For example, a microfluidic channel may be in fluid communication with a reaction chamber such that a fluid may flow freely into the reaction chamber from the microfluidic channel. The terms “in fluid communication” or “fluidically coupled” allow for two spatial regions being in fluid communication through one or more valves, restrictors, or other fluidic components to control or regulate a flow of fluid through a system.

As used herein, the term “immobilized,” when used with respect to a biomolecule or biological or chemical substance, includes substantially attaching the biomolecule or biological or chemical substance at a molecular level to a surface. For example, a biomolecule or biological or chemical substance may be immobilized to a surface of the substrate material using adsorption techniques including non-covalent interactions (e.g., electrostatic forces, van der Waals, and dehydration of hydrophobic interfaces) and covalent binding techniques where functional groups or linkers facilitate attaching the biomolecules to the surface. Immobilizing biomolecules or biological or chemical substances to a surface of a substrate material may be based upon the properties of the substrate surface, the liquid medium carrying the biomolecule or biological or chemical substance, and the properties of the biomolecules or biological or chemical substances themselves. In some cases, a substrate surface may be functionalized (e.g., chemically or physically modified) to facilitate immobilizing the biomolecules (or biological or chemical substances) to the substrate surface. The substrate surface may be first modified to have functional groups bound to the surface. The functional groups may then bind to biomolecules or biological or chemical substances to immobilize them thereon.

As used herein, the term “magnet,” includes permanent magnets and electromagnets.

In some examples, nucleic acids can be attached to a surface and amplified. Examples of such amplification are described in U.S. Pat. No. 7,741,463, entitled “Method of Preparing Libraries of Template Polynucleotides,” issued Jun. 22, 2010, the disclosure of which is incorporated by reference herein, in its entirety. In some cases, repeated rounds of extension (e.g., amplification) using an immobilized primer and primer in solution may provide multiple copies of the nucleic acid.

In particular examples, the assay protocols executed by the systems and methods described herein include the use of natural nucleotides and also enzymes that are configured to interact with the natural nucleotides. Natural nucleotides include, for example, ribonucleotides or deoxyribonucleotides. Natural nucleotides can be in the mono-, di-, or tri-phosphate form and can have a base selected from adenine (A), Thymine (T), uracil (U), guanine (G) or cytosine (C). It will be understood however that non-natural nucleotides, modified nucleotides, or analogs of the aforementioned nucleotides can be used.

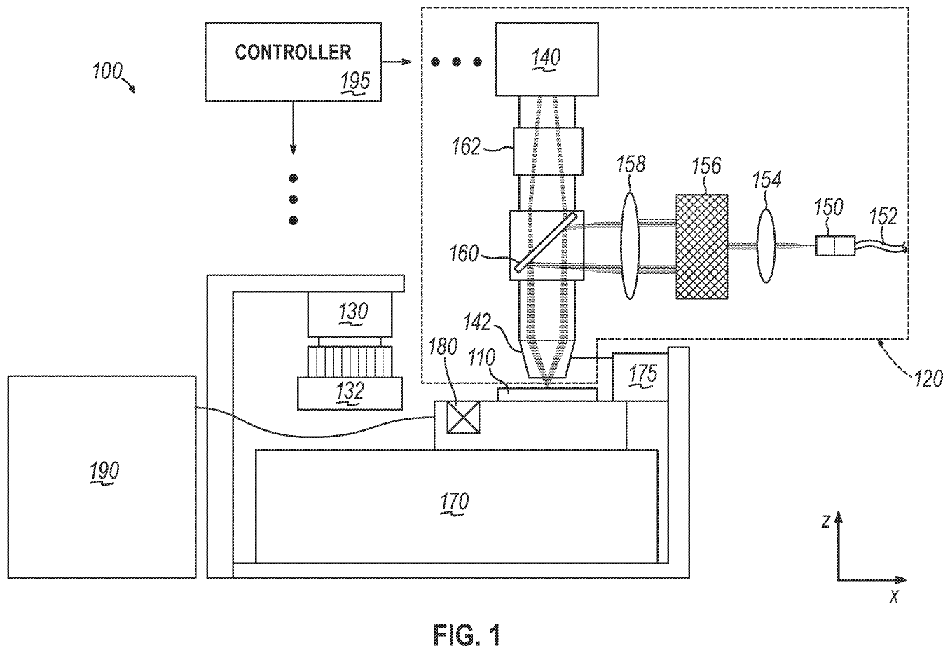

FIG. 1 depicts an example of components of a system (100) that may be used to provide biological or chemical analysis. In some examples, system (100) is a workstation that may be similar to a bench-top device. For example, a majority (or all) of the systems and components for conducting the designated reactions may be within a common housing. In particular examples, system (100) is a nucleic acid sequencing system (or sequencer) configured for various applications, including but not limited to de novo sequencing, resequencing of whole genomes or target genomic regions, and metagenomics. The sequencer may also be used for DNA or RNA analysis. In some versions, system (100) may also be configured to generate reaction sites in a flow cell (110). For example, system (100) may be configured to receive a sample and generate surface attached clusters of clonally amplified nucleic acids derived from the sample. In some implementations, the cluster may comprise a particular sample that is a distinguishable portion of the cluster even if the cluster is polyclonal as a result of one or more other samples being present within the cluster. System (100) is further configured to utilize an imaging assembly (120) to capture images of the reaction sites on flow cell (110).

In particular examples, the system (100) is to perform a large number of parallel reactions within flow cell (110). Flow cell (110) includes one or more reaction sites where designated reactions may occur. The reaction sites may be, for example, immobilized to a solid surface of flow cell (110) or immobilized to beads (or other movable substrates) that are located within corresponding reaction chambers of flow cell (110). The reaction sites may include, for example, clusters of clonally amplified nucleic acids. Flow cell (110) may include one or more flow channels that receive a solution from the system (100) and direct the solution toward the reaction sites. Optionally, flow cell (110) may engage a thermal element for transferring thermal energy into or out of the flow channel.

System (100) may include various components, assemblies, and systems (or sub-systems) that interact with each other to perform a predetermined method or assay protocol for biological or chemical analysis. For example, system (100) includes a system controller (195) that may communicate with the various components, assemblies, and sub-systems of the system (100). Examples of such components are described in greater detail below. Controller (195) may include one or more microprocessors, storage devices, and/or any other suitable electrical components that are configured to cooperate to execute control algorithms, data processing, etc.

In the present example, imaging assembly (120) includes a light emitter (150) that emits light that reaches reaction sites on flow cell (110). Light emitter (150) may include an incoherent light emitter (e.g., emit light beams output by one or more excitation diodes), or a coherent light emitter such as emitter of light output by one or more lasers or laser diodes. In the present example, light emitter (150) includes an optical fiber (152) for guiding an optical beam to be output via light emitter. However, other configurations of a light emitter (150) may be used. In some implementations, optical fiber (152) may optically couple to a plurality of different light sources (not shown), each light source emitting light of a different wavelength range. Although system (100) is illustrated as having a single light emitter (150), multiple light emitters (150) may be included in some other implementations.

In the present example, the light that is output from light emitter (150) is collimated by collimation lens (154). The collimated light is structured (patterned) by light structuring optical assembly (156) and reaches a projection lens (158). In some versions, projection lens (158) includes a lens element that is operable to translate along an axis (i.e., the axis on which light emitter (150), collimation lens (154), and light structuring optical assembly (156) are aligned) to adjust the structured beam shape and path. For example, projection lens (156) may be translated along this axis to account for a range of sample thicknesses (e.g., different cover glass thickness) of the sample in flow cell (110). In other implementations, projection lens (156) may be fixed and/or omitted and a moveable lens element may be positioned within a tube lens assembly in the emission optical path to account for focusing on an upper interior surface or lower interior surface of the flow cell (110) and/or spherical aberration introduced by movement of the objective lens assembly (142), The foregoing illumination components (150, 152, 154, 156, 158) are just examples. System (100) may alternatively include any other suitable components to provide illumination, in addition to or in lieu of any of the illumination components (150, 152, 154, 156, 158) described above. For instance, some other variations may omit structuring optical assembly (156) to use non-structured illumination and/or any other kind of illumination and/or optical arrangements (e.g., epifluorescence microscopy, etc.).

In the present example, the light directed by dichroic mirror (160) through objective lens assembly (142) onto a sample of a flow cell (110), which is positioned on a motion stage (170). In the case of fluorescent microscopy of a sample, a fluorescent element associated with the sample of interest fluoresces in response to the excitation light, and the resultant light is collected by objective lens assembly (142) and is directed to an image sensor of camera system (140) to detect the emitted fluorescence. As noted above, in some implementations, a tube lens assembly may be positioned between the objective lens assembly (142) and the dichroic mirror (160) or between the dichroic mirror (160) and the image sensor of the camera system (140). A moveable lens element may be translatable along a longitudinal axis of the tube lens assembly to account for focusing on an upper interior surface or lower interior surface of the flow cell (110) and/or spherical aberration introduced by movement of the objective lens assembly (142). In some implementations, a filter switching assembly (162) with one or more emission filters may be included, where the one or more emission filters may be used to pass through particular ranges of emission wavelengths and block (or reflect) other ranges of emission wavelengths. For example, the one or more emission filters may be used to direct different wavelength ranges of emitted light to different image sensors of the camera system (140) of imaging assembly (120). For instance, the emission filters may be implemented as dichroic mirrors that direct emission light of different wavelengths from flow cell (110) to different image sensors of camera system (140). In some variations, projection lens (158) is interposed between filter switching assembly (162) and camera system (140) instead of being positioned as shown in FIG. 1. Filter switching assembly (162) may be omitted in some versions.

In the example of system (100), fluid delivery module or device (190) may direct the flow of reagents (e.g., fluorescently labeled nucleotides, buffers, enzymes, cleavage reagents, etc.) to (and through) flow cell (110) and waste valve (180). Flow cell (110) may include one or more substrates upon which the samples are provided. For example, in the case of a system to analyze a large number of different nucleic acid sequences, flow cell (110) may include one or more substrates on which nucleic acids to be sequenced are bound, attached, or associated. The substrate may include any inert substrate or matrix to which nucleic acids may be attached, such as for example glass surfaces, plastic surfaces, latex, dextran, polystyrene surfaces, polypropylene surfaces, polyacrylamide gels, gold surfaces, and silicon wafers. In some applications, the substrate is within a channel or includes a channel formed within the substrate or other area at a plurality of locations formed in a matrix or array across the flow cell (110). System (100) may also include a temperature station actuator (130) and heater/cooler (132) that may optionally regulate the temperature of conditions of the fluids within the flow cell (110). In some implementations, the heater/cooler (132) may be fixed to a sample stage (170) upon which the flow cell (110) is placed and/or may be integrated therein to sample stage (170).

In particular implementations, the flow cell (110) may be implemented as a patterned flow cell including a transparent cover plate, a substrate, and configured to contain a liquid therebetween, and a biological sample may be located at an inside surface of the transparent cover plate and/or an inside surface of the substrate. The flow cell may include a large number (e.g., thousands, millions, or billions) of wells (also referred to as nanowells) or regions that are patterned into a defined array (e.g., a hexagonal array, rectangular array, etc.) into the substrate. Such wells may define reaction chambers providing reaction sites as described above. Each region may form a cluster (e.g., a monoclonal cluster, a substantially monoclonal cluster, or a polyclonal cluster) or more one than cluster) of a biological sample such as DNA, RNA, or another genomic material that may be sequenced, for example, using sequencing by synthesis. A substantially monoclonal cluster may be one where a particular sample forms a distinguishable portion of the cluster even if the cluster itself is polyclonal as a result of one or more other samples being present within the cluster. The flow cell may be further divided into a number of spaced apart lanes (e.g., eight lanes), each lane including a hexagonal array of clusters or a rectilinear array of clusters.

Flow cell (110) may be mounted on a sample stage (170) to provide movement and alignment of flow cell (110) relative to objective lens assembly (142). Sample stage (170) may have one or more actuators to allow sample stage (170) to move in any of three dimensions. For example, in terms of the Cartesian coordinate system, actuators may be provided to allow sample stage (170) to move in the x, y, and z directions relative to objective lens assembly (142), tilt relative to objective lens assembly (142), and/or otherwise move relative to objective lens assembly (142). Movement of sample stage (170) may allow one or more sample locations on flow cell (110) to be positioned in optical alignment with objective lens assembly (142). Movement of sample stage (170) relative to objective lens assembly (142) may be achieved by moving sample stage (170) itself, by moving objective lens assembly (142), by moving some other component of imaging assembly (120), by moving some other component of system (100), or any combination of the foregoing. For instance, in some implementations, the sample stage (170) may be actuatable in the X and Y directions relative to the objective lens assembly (142) while a focus component (175) or Z-stage may move the objective lens assembly (142) along the Z direction relative to the sample stage (170). Further implementations may also include moving imaging assembly (120) over a stationary flow cell (110). Thus, in some versions, flow cell (110) may be fixed during imaging while one or more components of imaging assembly (120) is/are moved to capture images at different regions of flow cell (110).

In some implementations, a focus component (175) may be included to control positioning of the objective lens relative to the flow cell (110) in the focus direction (e.g., along the z-axis or z-dimension). Focus component (175) may include one or more actuators physically coupled to the objective lens assembly (142), the optical stage, the sample stage (170), or a combination thereof, to move flow cell (110) on sample stage (170) relative to the objective lens assembly (142) to provide proper focusing for the imaging operation. In some implementations, the focus component (175) may utilize a focus tracking module (not shown) that is configured to detect a displacement of the objective lens assembly (142) relative to a portion of the flow cell (110) and output data indicative of an in-focus position to the focus component (175) to move the objective lens assembly (142) to position the corresponding portion of the flow cell (110) in focus of the objective lens assembly (142).

In some implementations, an actuator of the focus component (175) or for the sample stage (170) may be physically coupled to objective lens assembly (142), the optical stage, the sample stage (170), or a combination thereof, such as, for example, by mechanical, magnetic, fluidic, or other attachment or contact directly or indirectly to or with the stage or a component thereof. The actuator of the focus component (175) may be configured to move the objective lens assembly (142) in the z-direction while maintaining the sample stage (170) in the same plane (e.g., maintaining a level or horizontal attitude, perpendicular to the optical axis). In some implementations, the sample stage (170) includes an X direction actuator and a Y direction actuator to form an X-Y stage. The sample stage (170) may also be configured to include one or more tip or tilt actuators to tip or tilt sample stage (170) and/or a portion thereof, such as a flow cell chuck. This may be done, for example, so that flow cell (110) may be leveled dynamically to account for any slope in its surfaces.

Camera system (140) may include one or more image sensors to monitor and track the imaging (e.g., sequencing) of flow cell (110). Camera system (140) may be implemented, for example, as a CCD or CMOS image sensor camera, but other image sensor technologies (e.g., active pixel sensor) may be used. By way of further example only, camera system (140) may include a dual-sensor time delay integration (TDI) camera, a single-sensor camera, a camera with one or more two-dimensional image sensors, and/or other kinds of camera technologies. While camera system (140) and associated optical components are shown as being positioned above flow cell (110) in FIG. 1, one or more image sensors or other camera components may be incorporated into system (100) in numerous other ways as will be apparent to those skilled in the art in view of the teachings herein. For instance, one or more image sensors may be positioned under flow cell (110), such as within the sample stage (170) or below the sample stage (170), or may even be integrated into flow cell (110).

### II. Examples of Features to Modify Illumination Footprint at Flow Cell

As noted above, it may be desirable to provide uniformity in spatial irradiance from the excitation light emitted from imaging assembly (120) toward a flow cell (110), to thereby obtain spatial uniformity in the fluorescence emission signal collected by camera system (140). Otherwise, spatial irradiance non-uniformity in the illumination footprint illuminating reaction sites in flow cell (110) may ultimately lead to spatial signal variation of the fluorescence emission signal from fluorophores at the reaction sites of flow cell (110), as collected via camera system (140). Spatial signal variation of the fluorescence emission signal collected via camera system (140) may in turn cause lower accuracy of nucleotide sequencing information extracted from the imaging process. For instance, the fluorescence emission signal collected via camera system (140) may generally exhibit a dependence on position within an imaged field of view. Such dependence may be due to variation of excitation irradiance over the field of view. That is, if the illumination footprint at flow cell (110) has a higher intensity magnitude near the center and a lower intensity magnitude at the edges of the footprint, then the responsive emissions from fluorophores at the edges may similarly be lower in intensity when compared to those near the center. Some irradiation beam shaping features may tend to impart a periodic modulation on excitation “lines” that are mapped to the fields of view imaged onto the sensors of camera system (140).

Uniformity in spatial irradiance from the excitation light emitted from imaging assembly (120) may be achieved by reducing periodic modulation that may otherwise be found in the excitation light. In other words, it may be desirable to minimize modulation magnitude in the excitation light emitted from imaging assembly (120) toward a flow cell (110). As will be described in greater detail below, providing a rectangular shaped excitation illumination footprint may minimize the modulation magnitude in the excitation light, thereby maximizing spatial uniformity in the fluorescence emission signal collected by camera system (140).

To the extent that image processing techniques may be applied to images captured by camera system (140) through software (e.g., executed via controller (195)) to reduce the adverse effects from periodic modulation in the excitation light, it may be desirable to instead provide a hardware-based solution to prevent or at least reduce periodic modulation in the excitation light. Examples of hardware-based solutions to reduce or at least prevent periodic modulation in the excitation light will be described in greater detail below. After those examples of hardware-based solutions, examples of software-based solutions will be described. While the examples of hardware-based solutions and the examples of software-based solutions are described separately, there may be scenarios where one or more aspects of a hardware solution may be used in combination with one or more aspects of a software solution, such that the examples of hardware-based solutions and the examples of software-based solutions should not be viewed as being necessarily mutually exclusive.

### A. Examples of Different Optical Fiber Configurations

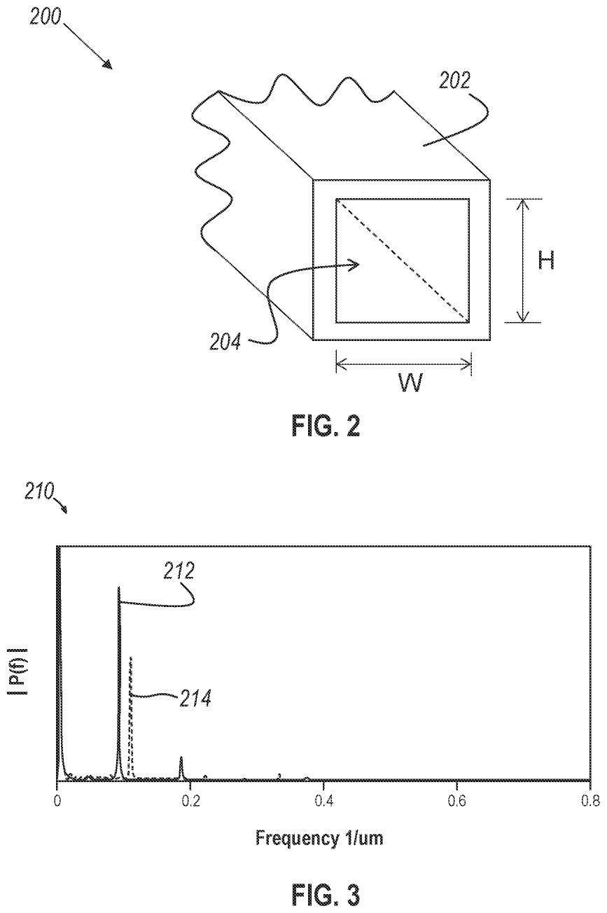

FIG. 2 shows an example of an optical fiber (200) that may be included in imaging assembly (120), as a variation of optical fiber (152) described above. Optical fiber (200) of this example includes a cladding (202) and a core (204). It should be understood that the depiction of optical fiber (200) in FIG. 2 is simplified, such that optical fiber (200) may include various other layers and components, including but not limited to a coating, strengthening features, a jacket, etc. Any suitable materials may be used to form cladding (202), core (204), and/or other features of optical fiber (200). In some versions, a laser diode (not shown) is optically coupled with optical fiber (200), such that core (204) is configured to transmit light generated by the laser diode. In some such versions, the laser diode includes a multimode laser diode; and optical fiber (200) is configured as a multimode fiber. By way of further example only, a multimode laser diode that is used with optical fiber (200) may include a laser diode that provides multimode light in one axis; and single mode light in another axis. Alternatively, any other suitable kind of light source (e.g., incoherent, etc.) may be optically coupled with optical fiber (200).

In the present example, core (204) has a square shaped cross-sectional profile, such that the height (H) and width (W) of core (204) are equal to each other. With core (204) having a square shaped cross-sectional profile, light emitted directly from optical fiber (200) may provide a square-shaped illumination footprint, which contains a substantially top hat (uniform) irradiance profile.

FIG. 3 shows a graph (210) with a first plot (212) and a second plot (214). First plot (212) represents an example of a modulation magnitude of a first wavelength range of excitation light (e.g., light in a green wavelength range) from optical fiber (200) as a function of frequency; while second plot (214) represents an example of a modulation magnitude of a second wavelength range of excitation light (e.g., light in a blue wavelength range) from optical fiber (200) as a function of frequency. As can be seen, plots (212, 214) include non-negligible spikes in modulation magnitude, which may indicate increased lack of uniformity in the spatial irradiance from the excitation light emitted from optical fiber (200). This may in turn indicate that there may be increased spatial signal variation of the fluorescence emission signal from fluorophores at the reaction sites of a flow cell (110) that is illuminated by excitation light from optical fiber (200).

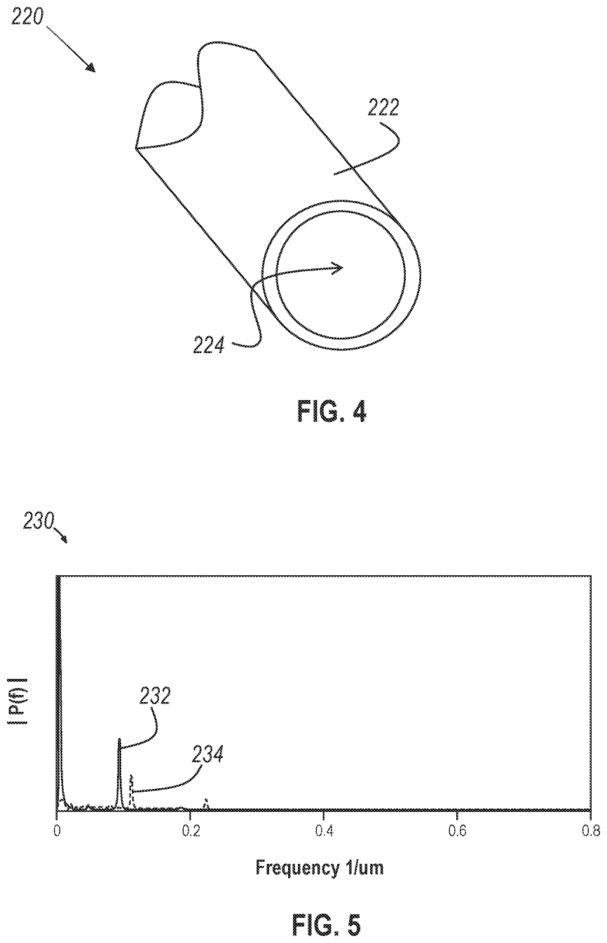

FIG. 4 shows another example of an optical fiber (220) that may be included in imaging assembly (120), as a variation of optical fiber (152) described above. Optical fiber (220) of this example includes a cladding (222) and a core (224). It should be understood that the depiction of optical fiber (200) in FIG. 2 is simplified, such that optical fiber (220) may include various other layers and components, including but not limited to a coating, strengthening features, a jacket, etc. Any suitable materials may be used to form cladding (222), core (224), and/or other features of optical fiber (220). In some versions, a laser diode (not shown) is optically coupled with optical fiber (220), such that core (224) is configured to transmit light generated by the laser diode. In some such versions, the laser diode includes a multimode laser diode; and optical fiber (220) is configured as a multimode fiber. By way of further example only, a multimode laser diode that is used with optical fiber (220) may include a laser diode that provides multimode light in one axis; and single mode light in another axis. Alternatively, any other suitable kind of light source (e.g., incoherent, etc.) may be optically coupled with optical fiber (220).

In the present example, core (224) has a circular shaped cross-sectional profile. With core (224) having a circular shaped cross-sectional profile, light emitted directly from optical fiber (220) may provide a circular-shaped illumination footprint, which contains a somewhat but not substantially top hat (uniform) irradiance profile.

FIG. 5 shows a graph (230) with a first plot (232) and a second plot (234). First plot (232) represents an example of a modulation magnitude of a first wavelength range of excitation light (e.g., light in a green wavelength range) from optical fiber (220) as a function of frequency; while second plot (234) represents an example of a modulation magnitude of a second wavelength range of excitation light (e.g., light in a blue wavelength range) from optical fiber (220) as a function of frequency. As can be seen, plots (232, 234) include non-negligible spikes in modulation magnitude, which may indicate increased lack of uniformity in the spatial irradiance from the excitation light emitted from optical fiber (220). This may in turn indicate that there may be increased spatial signal variation of the fluorescence emission signal from fluorophores at the reaction sites of a flow cell (110) that is illuminated by excitation light from optical fiber (220). To the extent that optical fiber (220) provides less modulation magnitude than optical fiber (200), it may be desirable to provide further reduced modulation magnitude.

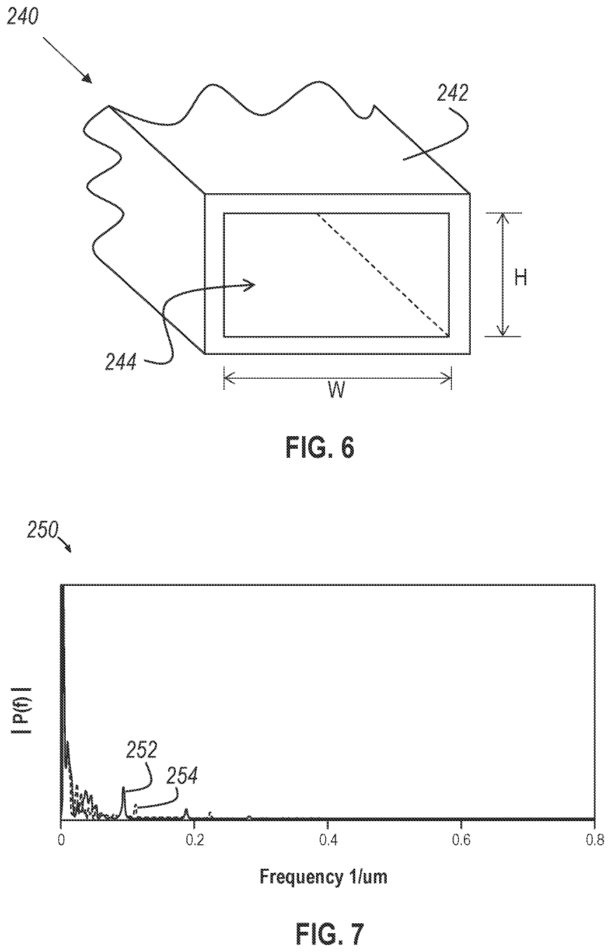

FIG. 6 shows another example of an optical fiber (240) that may be included in imaging assembly (120), as a variation of optical fiber (152) described above. Optical fiber (240) of this example includes a cladding (242) and a core (244). It should be understood that the depiction of optical fiber (240) in FIG. 2 is simplified, such that optical fiber (240) may include various other layers and components, including but not limited to a coating, strengthening features, a jacket, etc. Any suitable materials may be used to form cladding (242), core (244), and/or other features of optical fiber (240). In some versions, a laser diode (not shown) is optically coupled with optical fiber (240), such that core (244) is configured to transmit light generated by the laser diode. In some such versions, the laser diode includes a multimode laser diode; and optical fiber (240) is configured as a multimode fiber. By way of further example only, a multimode laser diode that is used with optical fiber (240) may include a laser diode that provides multimode light in one axis; and single mode light in another axis. Alternatively, any other suitable kind of light source (e.g., incoherent, etc.) may be optically coupled with optical fiber (240).

In the present example, core (244) has a rectangular shaped cross-sectional profile, such that the height (H) and width (W) of core (204) are not equal to each other. In some versions, the height (H) and width (W) of core (204) are configured such that core (204) has an aspect ratio of 2:1. In some other versions, the height (H) and width (W) of core (204) are configured such that core (204) has an aspect ratio of 3:1. In some other versions, the height (H) and width (W) of core (204) are configured such that core (204) has an aspect ratio of 4:1. Alternatively, the height (H) and width (W) of core (204) may be configured such that core (204) has some other aspect ratio based on the rectangular shape of core (204), including but not limited to an aspect ratio of greater than 4:1. By way of further example only, the width (W) of core (204) may be parallel to the pitch direction of light structuring optical assembly (156). In versions where lenslet arrays (380, 390) are used as described below, the width (W) of core (204) may be parallel to (or perpendicular to) the pitch direction of lenslet arrays (380, 390). Cladding (242) of the present example also has a rectangular shaped cross-sectional profile. With core (244) having a rectangular shaped cross-sectional profile, light emitted directly from optical fiber (240) may provide a rectangular-shaped illumination footprint, which contains a substantially top hat (uniform) irradiance profile.

FIG. 7 shows a graph (250) with a first plot (252) and a second plot (254). First plot (252) represents an example of a modulation magnitude of a first wavelength range of excitation light (e.g., light in a green wavelength range) from optical fiber (240) as a function of frequency; while second plot (254) represents an example of a modulation magnitude of a second wavelength range of excitation light (e.g., light in a blue wavelength range) from optical fiber (240) as a function of frequency. As can be seen, plots (252, 254) include relatively small spikes in modulation magnitude, which may indicate substantial uniformity in the spatial irradiance from the excitation light emitted from optical fiber (240). This may in turn indicate that there may be minimal or otherwise acceptable spatial signal variation of the fluorescence emission signal from fluorophores at the reaction sites of a flow cell (110) that is illuminated by excitation light from optical fiber (240).

Thus, in comparison to optical fibers (200, 220), optical fiber (240) may ultimately provide better performance in the context of system (100). This may be due, at least in part, to the rectangular shaped cross-sectional profile of core (244). In particular, the rectangular shaped cross-sectional profile of core (24) may provide a rectangular shaped illumination footprint in a far field plane (e.g., on a flow cell (110) or elsewhere) that is illuminated with light from optical fiber (240).

### B. Examples of Beam Shaping Features to Convert Illumination Footprint

As described above, it may be desirable to provide a rectangular shaped illumination footprint with the excitation light emitted from imaging assembly (120) toward a flow cell (110). As also described above, a rectangular shaped illumination footprint may be achieved by using an optical fiber (240) having a rectangular shaped core (244) as optical fiber (152) in imaging assembly (120). Alternatively, a rectangular shaped illumination footprint may be achieved using one or more beam shaping features positioned along the optical path between optical fiber (152) and flow cell (110). The following describes several examples of illuminating assemblies (300, 330, 360, 500) that provide beam shaping features configured and arranged to provide a rectangular shaped illumination footprint at flow cell (110).

It should be understood that the components of the illuminating assemblies (300, 330, 360, 500) described below may be incorporated into imaging assembly (120) in place of optical fiber (152), light emitter (150), collimation lens (154), light structuring optical assembly (156), and projection lens (158) shown in FIG. 1. Thus, the light from the illuminating assemblies (300, 330, 360, 500) described below may be directly communicated to dichroic mirror (160), and through objective lens assembly (142), to reach one or more reaction sites of flow cell (110). Alternatively, any other suitable optical component(s) (or no additional optical components) may be interposed between the illuminating assemblies (300, 330, 360, 500) described below and flow cell (110).

### 1. Example of Illuminating Assembly with Anamorphic Lens Assembly to Reshape Illumination Footprint

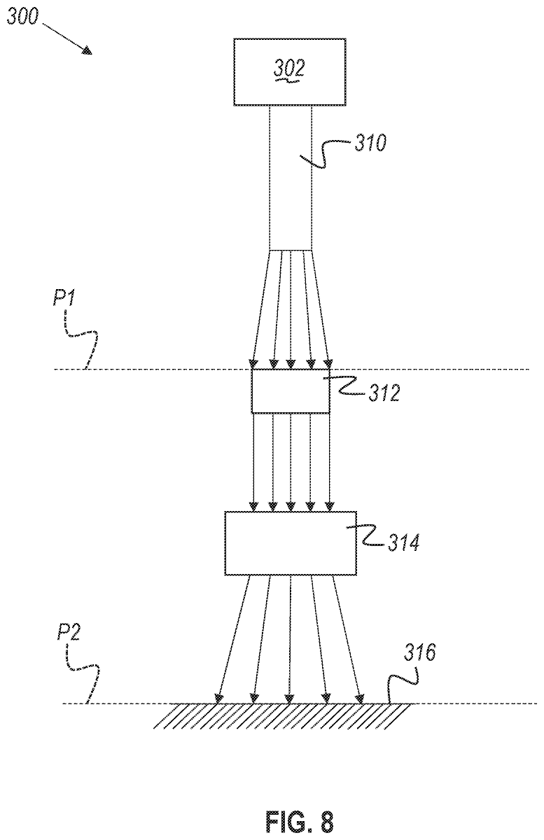

FIG. 8 shows an example of an illuminating assembly (300) that may be incorporated into imaging assembly (120), Illuminating assembly (300) of this example includes a light source (302), an optical fiber (310), a collimator (312), and a lens assembly (314). In some versions, light source (302) includes a laser diode. In some such versions, the laser diode includes a multimode laser diode; and optical fiber (310) is configured as a multimode fiber. By way of further example only, a multimode laser diode of light source (302) may include a laser diode that provides multimode light in one axis; and single mode light in another axis. Alternatively, light source (302) may be configured to provide incoherent light to optical fiber (310) and/or any other kind of light.

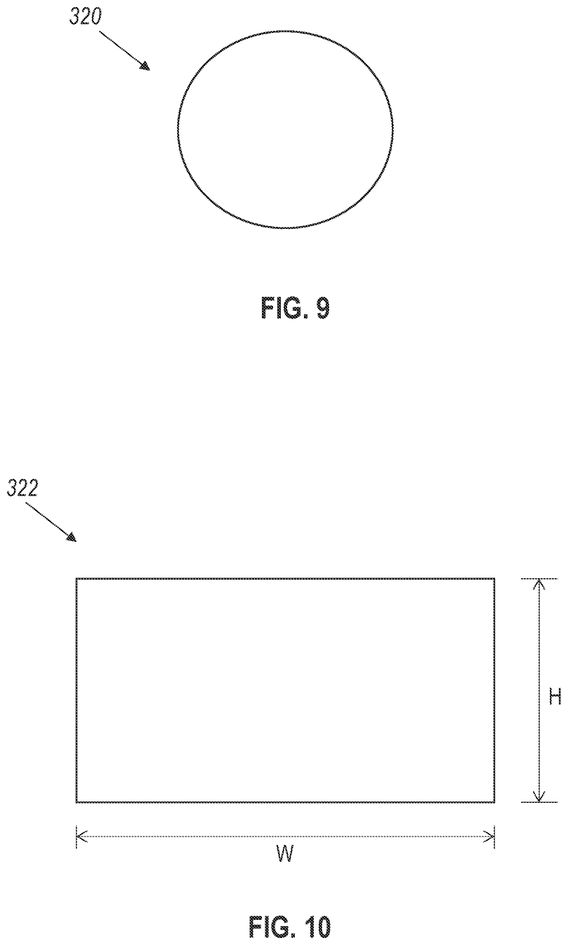

In the present example, optical fiber (310) has a circular shaped core, such that optical fiber (310) is configured like optical fiber (220). Alternatively, optical fiber (310) may have a square shaped core like optical fiber (200), a rectangular shaped core like optical fiber (240), or any other suitable configuration. Light emitted from optical fiber (310) reaches collimator (312) at a first plane (P1). In this example, the illumination footprint (320) at first plane (P1) has a circular shape, as shown in FIG. 9. It should be understood that even variations where optical fiber (310) is configured like optical fiber (200) or like optical fiber (240), illumination footprint (320) may still have some rounded aspect, though not necessarily purely circular. Collimator (312) collimates the light from optical fiber (310). By way of example only, collimator (312) may include a rotationally symmetric lens. Alternatively, collimator (312) may include a non-rotationally symmetric lens, may include any other suitable feature(s), and/or may take any other suitable form.

After passing through collimator (312), the light reaches lens assembly (314). By way of example only, lens assembly (314) may include an anamorphic lens assembly. Alternatively, lens assembly (314) may include a non-rotationally symmetric lens, may include any other suitable feature(s), and/or may take any other suitable form. Lens assembly (314) is optically configured to convert the illumination footprint into a rectangular shape. In particular, FIG. 8 shows an illuminated surface (316) at a second plane (P2). Illuminated surface (316) may correspond to surfaces of reaction sites in flow cell (110) (e.g., where the output of lens assembly (314) is directed through objective lens assembly (142) to reach flow cell (110) in an arrangement like the arrangement shown in FIG. 1). FIG. 10 shows illumination footprint (322) at second plane (P2) having a rectangular shape, such that the height (H) and width (W) of illumination footprint (322) are not equal to each other. In some versions, the width (W) of illumination footprint (322) is substantially greater than the height (H) of illumination footprint (322). In some such versions, illumination footprint has an aspect ratio that is greater than or equal to approximately 10:1. Alternatively, illumination footprint (322) may have any other suitable rectangular aspect ratio. Some variations may provide an illumination footprint (322) having an elliptical shape. As indicated above, the illumination footprint (322) may provide irradiance at surfaces of reaction sites in flow cell (110) (e.g., where the output of lens assembly (314) is directed through objective lens assembly (142) to reach flow cell (110) in an arrangement like the arrangement shown in FIG. 1).

Illuminating assembly (300) may thus yield an illumination output that is effectively the same as an output from an optical fiber (240) with a rectangular core (244), even if optical fiber (310) has a core that is circular, square, or otherwise shaped. In some variations, optical fiber (310) may have a rectangular core (e.g., like rectangular core (244) of optical fiber (240)), such that lens assembly (314) may further widen the aspect ratio of the illumination footprint. In other words, the rectangular illumination footprint at second plane (P2) may have a wider aspect ratio than the aspect ratio of a square or rectangular fiber core (204, 244), due to anamorphosis imposed by lens assembly (314).

The foregoing illuminating assembly (300) may be incorporated into the system (100) such that the illumination footprint (322) can be configured to correspond to a corresponding image sensor size and/or a predetermined dimensional size for illuminating a portion of the flow cell (110) while utilizing an optical fiber (310) that does not need to be design specifically for the illumination footprint (322).

### 2. Example of Illuminating Assembly with Anamorphic Collimator to Reshape Illumination Footprint

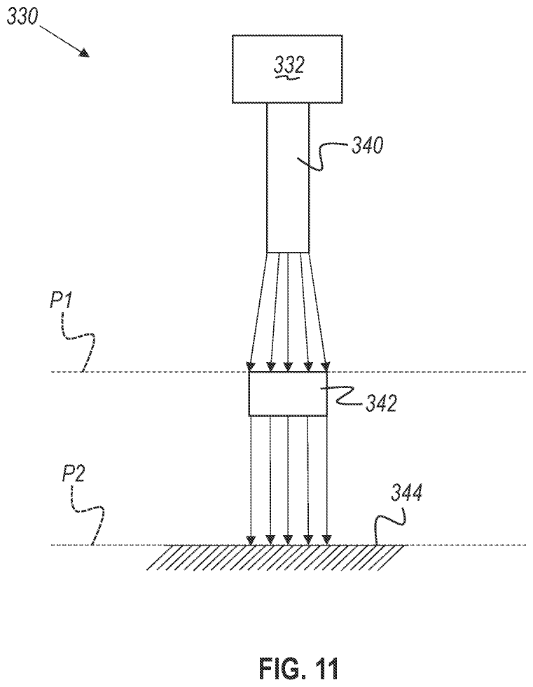

FIG. 11 shows another example of an illuminating assembly (330) that may be incorporated into imaging assembly (120). Illuminating assembly (330) of this example includes a light source (332), an optical fiber (340), and a collimator (342). In some versions, light source (332) includes a laser diode. In some such versions, the laser diode includes a multimode laser diode; and optical fiber (340) is configured as a multimode fiber. By way of further example only, a multimode laser diode of light source (332) may include a laser diode that provides multimode light in one axis; and single mode light in another axis. Alternatively, light source (332) may be configured to provide incoherent light to optical fiber (340) and/or any other kind of light.

In the present example, optical fiber (340) has a circular shaped core, such that optical fiber (340) is configured like optical fiber (220). Alternatively, optical fiber (340) may be configured like optical fiber (200) or have any other suitable configuration. Light emitted from optical fiber (340) reaches collimator (342) at a first plane (P1). In this example, the illumination footprint (not shown) at first plane (P1) has a circular shape, similar to illumination footprint (320) shown in FIG. 9.

Collimator (342) collimates the light from optical fiber (310). By way of example only, collimator (342) may include an anamorphic collimator, such that collimator (342) is optically configured to convert the illumination footprint (not shown) into a rectangular or elliptical shape on an illuminated surface (344) at a second plane (P2). Illuminated surface (344) may correspond to surfaces of reaction sites in flow cell (110). The illumination footprint on illuminated surface (344) at second plane (P2) may be similar to illumination footprint (322) shown in FIG. 10. Illuminating assembly (330) may thus yield an illumination output that is effectively the same as an output from an optical fiber (240) with a rectangular core (244), even if optical fiber (340) has a core that is circular, square, or otherwise shaped.

In some variations, optical fiber (340) may have a rectangular core (e.g., like rectangular core (244) of optical fiber (240)), such that collimator (342) may further widen the aspect ratio of the illumination footprint. In other words, the rectangular illumination footprint at second plane (P2) may have a wider aspect ratio than the aspect ratio of rectangular core (244), due to anamorphosis imposed by collimator (342). In some variations, light from collimator (342) also passes through an anamorphic lens assembly (e.g., like lens assembly (314)), such that the anamorphic lens assembly further emphasizes the anamorphosis imposed by collimator (342), thereby providing a further wider aspect ratio in the illumination footprint on illuminated surface (344) at second plane (P2).

While not shown in FIG. 8, illuminating assembly (330) may also include other optical components interposed between collimator (342) and illuminated surface (344), including but not limited to one or more microlens arrays and/or other optical features. Examples of other arrangements that include microlens arrays will be described in greater detail below.

The foregoing illuminating assembly (330) may be incorporated into the system (100) such that the illumination footprint at the illuminated surface (344) can be configured to correspond to a corresponding image sensor size and/or a predetermined dimensional size for illuminating a portion of the flow cell (110) while utilizing an optical fiber (340) that does not need to be design specifically for the illumination footprint at the illuminated surface (344).

### 3. Example of Illuminating Assembly with Diffuser and Microlens Arrays to Reshape Illumination Footprint

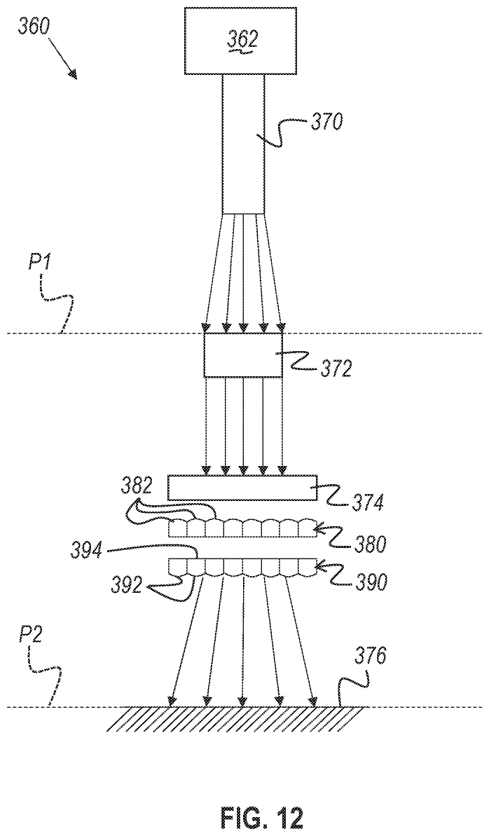

FIG. 12 shows another example of an illuminating assembly (360) that may be incorporated into imaging assembly (120). Illuminating assembly (360) of this example includes a light source (362), an optical fiber (370), a collimator (372), a diffuser (374), a first lenslet array (380), and a second lenslet array (390). In some versions, light source (362) includes a laser diode. In some such versions, the laser diode includes a multimode laser diode; and optical fiber (370) is configured as a multimode fiber. By way of further example only, a multimode laser diode of light source (362) may include a laser diode that provides multimode light in one axis; and single mode light in another axis. Alternatively, light source (362) may be configured to provide incoherent light to optical fiber (370) and/or any other kind of light.

In the present example, optical fiber (370) has a circular shaped core, such that optical fiber (370) is configured like optical fiber (220). Alternatively, optical fiber (370) may be configured like optical fiber (200) or have any other suitable configuration. Light emitted from optical fiber (370) reaches collimator (372) at a first plane (P1). In this example, the illumination footprint (not shown) at first plane (P1) has a circular shape, similar to illumination footprint (320) shown in FIG. 9. Collimator (372) collimates the light from optical fiber (370). By way of example only, collimator (372) may include an anamorphic collimator, like collimator (342) of illuminating assembly (330). As another example, collimator (372) may include a rotationally symmetric lens, like collimator (312) of illuminating assembly (300). In some versions where collimator (372) includes a rotationally symmetric lens, the light may pass through an anamorphic lens assembly, like lens assembly (314) of illuminating assembly (300), before reaching diffuser (374) as described below. Alternatively, collimator (372) may include any other suitable feature(s) and/or take any other suitable form.

After passing through collimator (372), the light reaches diffuser (374). Diffuser (374) is configured to increase the field angle of light entering lenslet arrays (380, 390). In the present example, diffuser (374) includes a one-dimensional diffuser, though some other versions may include a two-dimensional diffuser. The light exiting collimator (372) and reaching diffuser (374) may have a residual divergence related to the size of the core of optical fiber (370) divided by the focal length of collimator (372). In versions where diffuser (374) includes a one-dimensional diffuser, diffuser (374) increases the residual divergence in one direction. In other words, diffuser (374) increases the field angle of the light.

After passing through diffuser (374), the light reaches first lenslet array (380); then second lenslet array (390). Each lenslet array (380, 390) includes a respective plurality of lenslets (382, 392). In some versions, lenslet arrays (380, 390) are configured substantially identically to each other, but face in opposite directions. For instance, lenslets (382) of first lenslet array (380) may be configured and arranged substantially identically to the configuration and arrangement of lenslets (392) of second lenslet array (390). By way of further example only, lenslet arrays (380, 390) may be separated from each other by a distance that is approximately equal to the focal length of a lenslet (382, 392). Lenslet arrays (380, 390) might also represent optical components that have arrays on two surfaces. In some versions, including but not limited to versions where camera system (140) includes time delay integration (TDI) imaging components, each lenslet array (380, 390) includes cylindrical microlens array surfaces (CuLAs). Such CuLAs may fan light beams out in one direction. In such versions, a one-dimensional version of diffuser (374) may be positioned and configured to increase the fill of the CuLA lenslets in the pitch direction, without increasing the fill of the CuLA lenslets in the non-pitch direction. While some versions of lenslet arrays (380, 390) include cylindrical mircolens arrays, some other versions of lenslet arrays (380, 390) may include two-dimensional lenslet arrays. Alternatively, lenslet arrays (380, 390) may have any other suitable configuration.

As the light passes through first lenslet array (380), each lenslet (382) of the first lenslet array (380) causes an image of the core of optical fiber (370) to form proximate to the active surface (394) of second lenslet array (394). The size of this optical fiber (370) core image may be proportional to the apparent size of optical fiber (370) core. Likewise, the size of this optical fiber (370) core image proximate to the active surface (394) of second lenslet array (394) may be proportional to the residual divergence of the light. In some cases, as the apparent size of optical fiber (370) core increases, the fill factor of second lenslet array (390) may correspondingly increase. This fill factor may be understood as the ratio of the active refracting area (i.e., the area of a lenslet (392) of second lenslet array (390) that directs the light to illuminated surface (376)) to the total contiguous area occupied by a lenslet (392) of second lenslet array (390). As the fill factor of lenslets (392) increases, the modulation depth of the light exiting second lenslet array (390) may decrease.

The light exiting second lenslet array (390) provides an illumination footprint (not shown) in a rectangular shape on an illuminated surface (376) at a second plane (P2) far from second lenslet array (390). Illuminated surface (376) may correspond to surfaces of reaction sites in flow cell (110) (e.g., where the output of illuminating assembly (360) is directed through objective lens assembly (142) to reach flow cell (110) in an arrangement like the arrangement shown in FIG. 1). The illumination footprint on illuminated surface (376) at second plane (P2) may be similar to illumination footprint (322) shown in FIG. 10. The combination of collimator (372), diffuser (374), and lenslet arrays (380, 390) may effectively generate an illuminated footprint at second plane (P2), of illuminated surface (376), that is substantially larger than the footprint at first plane (PI). In other words, the collimator (372), diffuser (374), and lenslet arrays (380, 390) may provide an illumination output that is like the illumination output of a light source that is much larger than optical fiber (370), which may in turn provide substantially reduced modulation than would otherwise be provided in light communicated directly from optical fiber (370) in the absence of collimator (372), diffuser (374), and lenslet arrays (380, 390). Moreover, illuminating assembly (360) may yield an illumination output that is effectively the same as an output from an optical fiber (240) with a rectangular core (244), even if optical fiber (370) has a core that is circular, square, or otherwise shaped. Some versions of illuminating assembly (360) may also utilize an optical fiber that has a rectangular core, like rectangular core (244) of optical fiber (240).

The foregoing illuminating assembly (360) may be incorporated into the system (100) such that the illumination footprint at the illuminated surface (376) can be configured to correspond to a corresponding image sensor size and/or a predetermined dimensional size for illuminating a portion of the flow cell (110) while utilizing an optical fiber (370) that does not need to be design specifically for the illumination footprint at the illuminated surface (376).

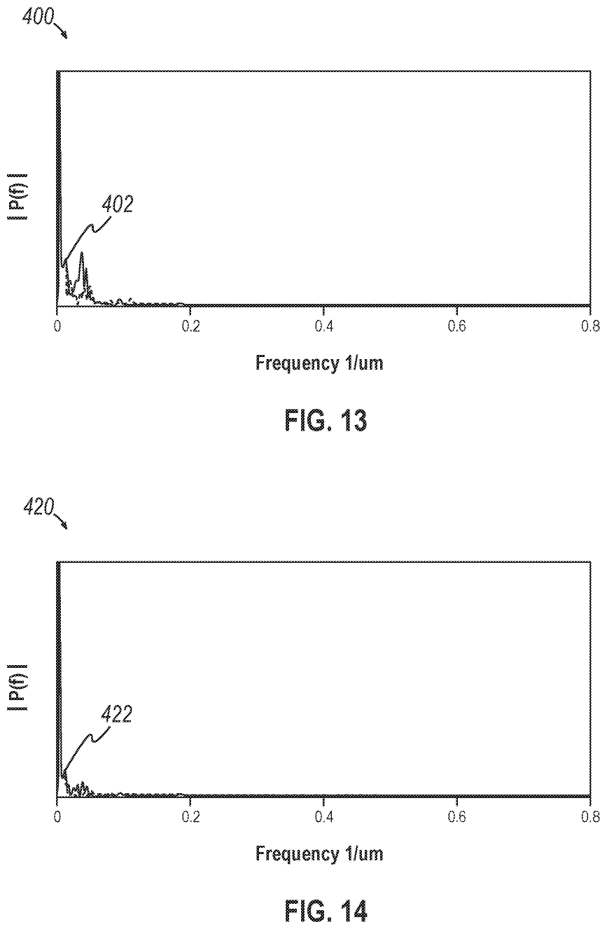

FIG. 13 shows a graph (400) with a plot (402) representing an example of a modulation magnitude of excitation light as a function of frequency, from a version of illuminating assembly (360) where optical fiber (200) is used as optical fiber (370) such that the illumination footprints at the foci of the lenslets (382) of first lenslet array (380) are square. Plot (402) may be understood to represent modulation in the illumination footprint at second plane (P2), which would be associated with reaction sites in flow cell (110). As can be seen, plot (402) has a relatively small spike in modulation magnitude. However, this spike is substantially smaller than the spikes seen in plots (212, 214) of FIG. 3, which may be understood to correspond with modulation in the illumination footprint at first plane (P1) when optical fiber (200) is used as optical fiber (370) in illuminating assembly (360).

The substantially smaller spike in modulation magnitude in plot (402) as compared to the spikes in plots (212, 214) may indicate how collimator (372), diffuser (374), and lenslet arrays (380, 390) together ultimately reduce modulation in the illumination footprint from optical fiber (200). Thus, collimator (372), diffuser (374), and lenslet arrays (380, 390) may together provide better uniformity in the spatial irradiance of reaction sites in flow cell (110), which may provide reduced spatial signal variation of the fluorescence emission signal from fluorophores at the reaction sites of a flow cell (110) that is illuminated by excitation light from illuminating assembly (360). Only one plot (402) is shown in FIG. 13, representing only one channel of excitation light. However, substantially similar results may be obtained for other channels of excitation light from optical fiber (370) in illuminating assembly (360).

FIG. 14 shows a graph (420) with a plot (422) representing an example of a modulation magnitude of excitation light as a function of frequency, from a version of illuminating assembly (360) where optical fiber (220) is used as optical fiber (370) such that the illumination footprints at the foci of lenslets (382) of first lenslet array (380) are circular. Plot (422) may be understood to represent modulation in the illumination footprint at second plane (P2), which would be associated with reaction sites in flow cell (110). As can be seen, plot (422) has a relatively small spike in modulation magnitude. However, this spike is substantially smaller than the spikes seen in plots (232, 234) of FIG. 5, which may be understood to correspond with modulation in the illumination footprint at first plane (P1) when optical fiber (220) is used as optical fiber (370) in illuminating assembly (360).

The substantially smaller spike in modulation magnitude in plot (422) as compared to the spikes in plots (232, 234) may indicate how collimator (372), diffuser (374), and lenslet arrays (380, 390) together ultimately reduce modulation in the illumination footprint from optical fiber (220). Thus, collimator (372), diffuser (374), and lenslet arrays (380, 390) may together provide better uniformity in the spatial irradiance of reaction sites in flow cell (110), which may provide reduced spatial signal variation of the fluorescence emission signal from fluorophores at the reaction sites of a flow cell (110) that is illuminated by excitation light from illuminating assembly (360). Only one plot (422) is shown in FIG. 14, representing only one channel of excitation light. However, substantially similar results may be obtained for other channels of excitation light from optical fiber (370) in illuminating assembly (360).

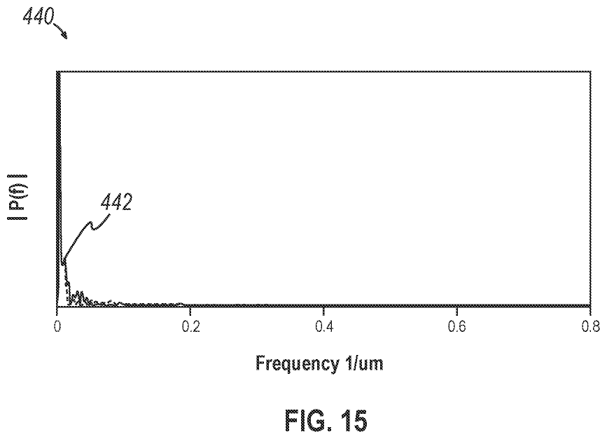

FIG. 15 shows a graph (440) with a plot (442) representing an example of a modulation magnitude of excitation light as a function of frequency, from a version of illuminating assembly (360) where optical fiber (240) is used as optical fiber (370) such that the illumination footprints at the foci of lenslets (382) of first lenslet array (380) are circular. Plot (442) may be understood to represent modulation in the illumination footprint at second plane (P2), which would be associated with reaction sites in flow cell (110). As can be seen, plot (442) has a relatively small spike in modulation magnitude. However, this spike is substantially smaller than the spikes seen in plots (252, 254) of FIG. 7, which may be understood to correspond with modulation in the illumination footprint at first plane (P1) when optical fiber (240) is used as optical fiber (370) in illuminating assembly (360).

The substantially smaller spike in modulation magnitude in plot (42) as compared to the spikes in plots (252, 254) may indicate how collimator (372), diffuser (374), and lenslet arrays (380, 390) together ultimately reduce modulation in the illumination footprint from optical fiber (240). Thus, collimator (372), diffuser (374), and lenslet arrays (380, 390) may together provide better uniformity in the spatial irradiance of reaction sites in flow cell (110), which may provide reduced spatial signal variation of the fluorescence emission signal from fluorophores at the reaction sites of a flow cell (110) that is illuminated by excitation light from illuminating assembly (360). Only one plot (442) is shown in FIG. 15, representing only one channel of excitation light. However, substantially similar results may be obtained for other channels of excitation light from optical fiber (370) in illuminating assembly (360).

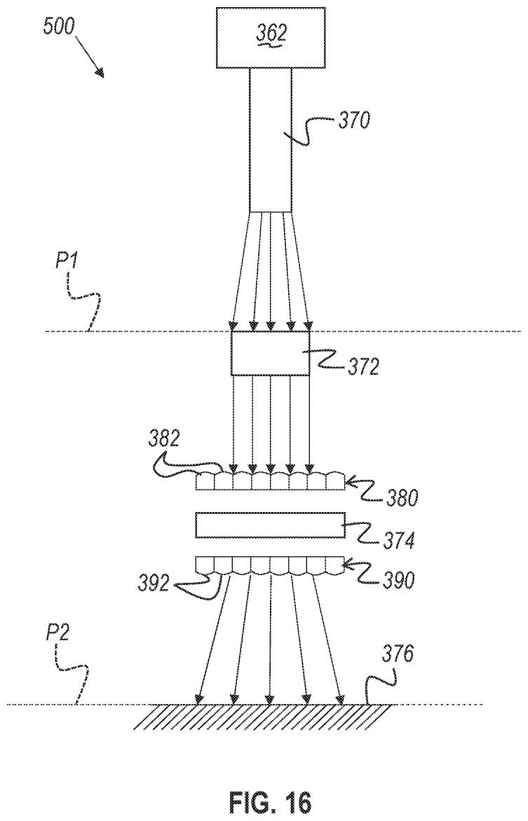

FIG. 16 shows another example of an illuminating assembly (500) that may be incorporated into imaging assembly (120). Illuminating assembly (500) of this example is a variation of illuminating assembly (360), such that illuminating assembly (500) includes the same components of illuminating assembly (360) but in a different arrangement. In particular, illuminating assembly (500) includes light source (362), optical fiber (370), collimator (372), diffuser (374), first lenslet array (380), and second lenslet array (390). However, unlike the arrangement of imaging assembly (360) where diffuser (374) is positioned between collimator (372) and first lenslet array (380), diffuser (374) is positioned between first lenslet array (380) and second lenslet array (390) in illuminating assembly (500). This arrangement of diffuser (374) between first lenslet array (380) and second lenslet array (390) in illuminating assembly (500) may yield substantially the same results as the different arrangement in illuminating assembly (360). In other words, illuminating assembly (500) may provide a rectangular shaped illumination footprint on illuminated surface (376) at second plane (P2), with substantially reduced modulation magnitude.

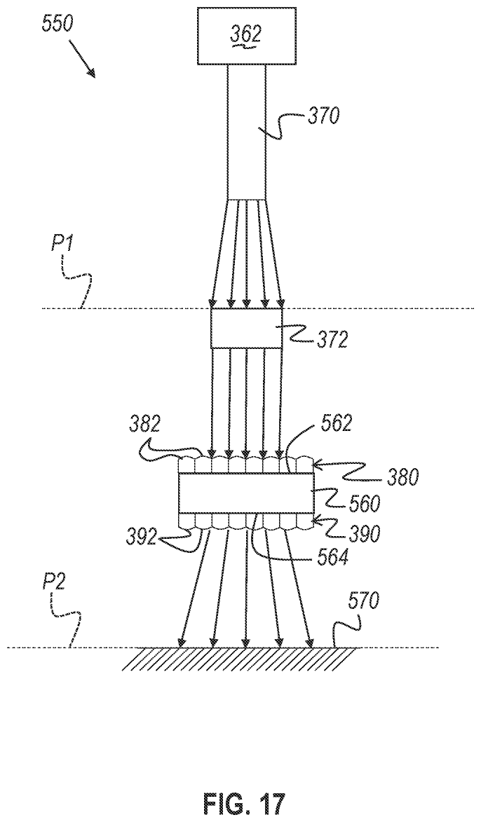

FIG. 17 shows another example of an illuminating assembly (550) that may be incorporated into imaging assembly (120). Illuminating assembly (550) of this example is a variation of illuminating assembly (500), such that illuminating assembly (500) includes most of the same components of illuminating assembly (500), except that illuminating assembly (500) includes a refractive substrate (560) instead of diffuser (374). Like illuminating assembly (500), illuminating assembly (550) of this example further includes light source (362), optical fiber (370), collimator (372), first lenslet array (380), and second lenslet array (390). Refractive substrate (560) of this example includes a first surface (562) and a second surface (564). First lenslet array (380) is disposed on first surface (562). Second lenslet array (390) is disposed on first surface (564). Refractive substrate (560) is thus directly interposed between lenslet arrays (380, 390) in this example.

Refractive substrate (560) is configured such that refractive substrate (560) will substantially focus a beam that is incident on first lenslet array (380) onto second lenslet array (390). This focusing aspect of refractive substrate (560) may be due in part to the thickness of refractive substrate (i.e., the distance between surfaces (562, 564), among other parameters. The arrangement of refractive substrate (560) between first lenslet array (380) and second lenslet array (390) in illuminating assembly (550) may yield substantially the same results as the different arrangement in illuminating assemblies (360, 500). In other words, illuminating assembly (500) may provide a rectangular shaped illumination footprint on illuminated surface (570) at second plane (P2), with substantially reduced modulation magnitude.

### C. Examples of Image Processing Techniques

In some scenarios, image processing techniques may be applied to images captured by camera system (140) through software (e.g., executed via controller (195)) to reduce the adverse effects from periodic modulation in the excitation light. For instance, the elements of each column of an image captured by camera system (140) may be summed, such that the reading of the pixel that is in the first row and first column is added to the reading of the pixel that is in the second row and first column. This may be done over all rows for each column. By way of further example only, for an image that is 5120 columns wide and 4096 rows tall, 4096 rows of each column may be summed to generate a one-dimensional array that has 5120 elements (i.e., as many elements as there were columns). These numbers are just examples, as an image may have any other suitable number of columns and rows.

The one-dimensional array described above may be referred to as a flat-fielding array. This flat-fielding array may be normalized by dividing each element of the array by the sum of the elements of the array. The flat-fielding array could further be normalized by dividing every element by the average of the flat-fielding array elements, after which the average of the flat-fielding elements would be 1.

This normalized array may be defined as a flat-fielding profile. Each row of the original image captured by camera system (140) may be divided by the flat-fielding profile. This process may render the modulation from the original image substantially imperceptible. Modulation may also be removed via frequency domain filtering. For instance, such filtering may utilize a high-pass filter or a notch filter that does not pass the spatial frequency of the modulation. In other words, in the event that one or more of the images has spatial patterns with specific periods, one or more frequency domain filters may be applied to reduce the spatial patterns.

The above-described image processing techniques may be used in versions of system (100) that incorporate at least one of optical fibers (200, 220, 240) in imaging assembly (120); in versions of system (100) that incorporate one of illuminating assemblies (300, 330, 360, 500) in imaging assembly (120); and/or in other versions of system (100). In some versions, the above-described image processing techniques are used to computationally reduce periodic modulation in images captured by camera system (140) when reaction sites in flow cell (110) are illuminated with a modulated excitation light. Thus, the above-described image processing techniques may serve as an alternative to a hardware-based solution (e.g., optical fiber (240), illuminating assemblies (300, 330, 360, 500), etc. ) to address modulation.

### III. Examples of Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus, comprising: a sequencing stage configured to receive a flow cell comprising a plurality of reaction sites, each reaction site being configured to contain a biological sample; and an illumination assembly configured to project light toward the sequencing stage to thereby illuminate the reaction sites, the illumination assembly including: a light source, a first lenslet array interposed between the light source and the sequencing stage, a second lenslet array interposed between the light source and the sequencing stage, and a diffuser interposed between the light source and the sequencing stage.

### Example 2

The apparatus of Example 1, the diffuser comprising a one-dimensional diffuser.

### Example 3

The apparatus of any of Examples 1 through 2, the first lenslet array comprising a cylindrical mircolens array.

### Example 4

The apparatus of any of Examples 1 through 3, the diffuser being interposed between the light source and the first lenslet array.

### Example 5

The apparatus of any of Examples 1 through 3, the diffuser being interposed between the first lenslet array and the second lenslet array.

### Example 6

The apparatus of any of Examples 1 through 3, the second lenslet array being interposed between the first lenslet array and the diffuser.

### Example 7

The apparatus of any of Examples 1 through 6, further comprising a collimator interposed between the light source and one or both of the first lenslet array or the diffuser.

### Example 8

The apparatus of Example 7, the collimator comprising an anamorphic collimator.

### Example 9

The apparatus of any of Examples 1 through 8, the first lenslet array comprising a plurality of lenslets, each lenslet having a focal length, the first lenslet array and the second lenslet array being spaced apart from each other by a distance approximately equal to the focal length.

### Example 10

The apparatus of any of Examples 1 through 9, the light source comprising an optical fiber.

### Example 11

The apparatus of Example 10, the optical fiber having a core with a square cross-sectional shape.

### Example 12

The apparatus of Example 10, the optical fiber having a core with a circular cross-sectional shape.

### Example 13

The apparatus of Example 10, the optical fiber having a core with a rectangular cross-sectional shape defined by a length and a width, the width being greater than the length.

### Example 14

The apparatus of Example 13, the length and width providing an aspect ratio of at least 2:1.

### Example 15

The apparatus of Example 13, the length and width providing an aspect ratio of at least 3:1.

### Example 16

The apparatus of Example 13, the length and width providing an aspect ratio of at least 4:1.

### Example 17

The apparatus of any of Examples 10 through 16, the light source comprising a laser diode.

### Example 18

The apparatus of any of Examples 10 through 17, the laser diode comprising a multimode diode, the optical fiber being configured to transmit multiple channels of light.

### Example 19

The apparatus of any of Examples 1 through 18, further comprising a camera system, the camera system being operable to capture images of the reaction sites.

### Example 20

The apparatus of Example 19, the camera system including a time delay integration camera.

### Example 21

The apparatus of any of Examples 19 through 20, the camera system being operable to capture light emitted by fluorophores at the reaction sites in response to the light projected by the illumination assembly.

### Example 22

A method comprising: communicating light through an illumination assembly toward a sequencing stage, the illumination assembly including: a light source, a first lenslet array interposed between the light source and the sequencing stage, a second lenslet array interposed between the light source and the sequencing stage, and a diffuser interposed between the light source and the sequencing stage; the communicated light being further communicated toward a plurality of reaction sites at the sequencing stage, each reaction site containing a biological sample.

### Example 23

The method of Example 22, the diffuser comprising a one-dimensional diffuser.

### Example 24

The method of any of Examples 22 through 23, the first lenslet array comprising a cylindrical mircolens array.

### Example 25

The method of any of Examples 22 through 24, the diffuser being interposed between the light source and the first lenslet array.

### Example 26

The method of any of Examples 22 through 24, the diffuser being interposed between the first lenslet array and the second lenslet array.

### Example 27

The method of any of Examples 22 through 24, the second lenslet array being interposed between the first lenslet array and the diffuser.

### Example 28

The method of any of Examples 22 through 27, the illumination assembly further comprising a collimator interposed between the light source and one or both of the first lenslet array or the diffuser.

### Example 29

The method of Example 26, the collimator comprising an anamorphic collimator.

### Example 30

The method of any of Examples 22 through 29, the first lenslet array comprising a plurality of lenslets, each lenslet having a focal length, the first lenslet array and the second lenslet array being spaced apart from each other by a distance approximately equal to the focal length.

### Example 31

The method of any of Examples 22 through 30, the light source comprising an optical fiber.

### Example 32

The method of Example 31, the optical fiber having a core with a square cross-sectional shape.

### Example 33

The method of Example 31, the optical fiber having a core with a circular cross-sectional shape.

Example 34

The method of Example 31, the optical fiber having a core with a rectangular cross-sectional shape defined by a length and a width, the width being greater than the length.

### Example 35

The method of Example 34, the length and width providing an aspect ratio of at least 2:1.

### Example 36

The method of Example 34, the length and width providing an aspect ratio of at least 3:1.

### Example 37

The method of Example 34, the length and width providing an aspect ratio of at least 4:1

### Example 38

The method of any of Examples 31 through 37, the light source comprising a laser diode.

### Example 39

The method of any of Examples 31 through 38, the laser diode comprising a multimode diode, the optical fiber being configured to transmit multiple channels of light.

### Example 40

The method of any of Examples 22 through 39, further comprising capturing images of the reaction sites with a camera system.

### Example 41

The method of Example 40, the camera system including a time delay integration camera.

### Example 42

The method of any of Examples 40 through 41, the captured images including an original image having rows and columns, the method further comprising: summing the rows of each column of the original image to form a one-dimensional flat-fielding array having a plurality of elements; and dividing each column of the original image by the flat-fielding array to yield a demodulated image.

### Example 43

The method of any of Examples 40 through 42, the reaction sites including fluorophores, the fluorophores emitting light in response to the communicated light reaching the reaction sites, the capturing images of the reaction sites with the camera system including capturing the light emitted by the fluorophores at the reaction sites.

### Example 44

The method of Example 43, further comprising identifying at least one nucleotide at a reaction site of the plurality of reaction sites, based on the captured light emitted by at least one fluorophore at the reaction site.

### Example 45

The method of any of Examples 22 through 44, the reaction sites being located in a flow cell.

### Example 46

The method of Example 45, further comprising performing sequencing by synthesis on the flow cell.

### Example 47

An apparatus, comprising: a sequencing stage configured to receive a flow cell comprising a plurality of reaction sites, each reaction site being configured to contain a biological sample; and an illumination assembly configured to project light toward the sequencing stage to thereby illuminate the reaction sites, the illumination assembly including: a light source, a collimator interposed between the light source and the sequencing stage, an anamorphic lens assembly interposed between the collimator and the sequencing stage.

### Example 48

The apparatus of Example 47, the collimator comprising a rotationally symmetric lens.

### Example 49

The apparatus of any of Examples 47 through 48, the anamorphic lens assembly being configured to convert an illumination footprint from the light source to a rectangular shape.

### Example 50

The apparatus of Example 49, the light source comprising an optical fiber having a core with a square cross-sectional shape.

### Example 51

The apparatus of Example 49, the light source comprising an optical fiber having a core with a circular cross-sectional shape.

### Example 52

The apparatus of any of Examples 49 through 51, the rectangular shape having an aspect ratio of at least 10:1.

### Example 53

The apparatus of any of Examples 47 through 48, the anamorphic lens assembly being configured to convert an illumination footprint from the light source to an elliptical shape.

### Example 54

The apparatus of Example 53, the elliptical shape having an aspect ratio of at least 10:1.

### Example 55

The apparatus of any of Examples 49 through 54, the anamorphic lens assembly being configured to provide the illumination footprint at the reaction sites.

### Example 56

An apparatus, comprising: a sequencing stage configured to receive a flow cell comprising a plurality of reaction sites, each reaction site being configured to contain a biological sample; and an illumination assembly configured to project light toward the sequencing stage to thereby illuminate the reaction sites, the illumination assembly including: a light source, and an anamorphic collimator interposed between the light source and the sequencing stage.

### Example 57

The apparatus of any of Example 56, the anamorphic lens assembly being configured to convert an illumination footprint from the light source to a rectangular shape.

### Example 58

The apparatus of Example 57, the light source comprising an optical fiber having a core with a square cross-sectional shape.

### Example 59

The apparatus of Example 57, the light source comprising an optical fiber having a core with a circular cross-sectional shape.

### Example 60

The apparatus of any of Examples 57 through 59, the rectangular shape having an aspect ratio of at least 10:1.

### Example 61

The apparatus of any of Example 56, the anamorphic lens assembly being configured to convert an illumination footprint from the light source to an elliptical shape.

### Example 62

The apparatus of Example 61, the elliptical shape having an aspect ratio of at least 10:1.

### Example 63

The apparatus of any of Examples 56 through 62, the anamorphic lens assembly being configured to provide the illumination footprint at the reaction sites.

### Example 64

A method comprising: communicating light through an illumination assembly toward a plurality of reaction sites at a sequencing stage, each reaction site containing a biological sample; capturing images of the reaction sites with a camera system, the camera system including a time delay integration camera, the captured images including an original image having rows and columns; summing the rows of the original image to form a one-dimensional flat-fielding array having a plurality of elements; and dividing each column of the original image by the flat-fielding array to yield a demodulated image.

### Example 65

The method of Example 64, the demodulated image having spatial patterns with specific periods, the method further comprising applying one or more frequency domain filters to reduce the spatial patterns.

### Example 66

A method comprising: communicating light through an illumination assembly toward a plurality of reaction sites at a sequencing stage, each reaction site containing a biological sample; capturing images of the reaction sites with a camera system, the camera system including a time delay integration camera, the captured images including an original image having spatial patterns with specific periods; and applying one or more frequency domain filters to reduce the spatial patterns and thereby yield a demodulated image.

### Example 67

An apparatus, comprising: a sequencing stage configured to receive a flow cell comprising a plurality of reaction sites, each reaction site being configured to contain a biological sample; and an illumination assembly configured to project light toward the sequencing stage to thereby illuminate the reaction sites, the illumination assembly including: a light source, a first lenslet array interposed between the light source and the sequencing stage, and a second lenslet array interposed between the light source and the sequencing stage.

### Example 68

The apparatus of Example 67, further comprising a diffuser interposed between the light source and the sequencing stage.

### Example 69

The apparatus of Example 68, the diffuser being interposed between the light source and the first lenslet array.

### Example 70

The apparatus of Example 68, the diffuser being interposed between the first lenslet array and the second lenslet array.

### Example 71

The apparatus of any of Example 68, the second lenslet array being interposed between the first lenslet array and the diffuser.

### Example 72

The apparatus of any of Examples 68 through 71, further comprising a collimator interposed between the light source and one or both of the first lenslet array or the diffuser.

### Example 73

The apparatus of Example 72, the collimator comprising an anamorphic collimator.

### Example 74

The apparatus of any of Examples 67 through 73, the first lenslet array comprising a cylindrical mircolens array.

### Example 75

The apparatus of any of Examples 67 through 74, the first lenslet array comprising a plurality of lenslets, each lenslet having a focal length, the first lenslet array and the second lenslet array being spaced apart from each other by a distance approximately equal to the focal length.

### Example 76

The apparatus of any of Examples 1 through 78, the light source comprising an optical fiber.

### Example 77

The apparatus of Example 76, the optical fiber having a core with a square cross-sectional shape.

### Example 78

The apparatus of Example 76, the optical fiber having a core with a circular cross-sectional shape.

### Example 79

The apparatus of Example 76, the optical fiber having a core with a rectangular cross-sectional shape defined by a length and a width, the width being greater than the length.

### Example 80

The apparatus of Example 79, the length and width providing an aspect ratio of at least 2:1.

### Example 81

The apparatus of Example 79, the length and width providing an aspect ratio of at least 3:1.

### Example 82

The apparatus of Example 79, the length and width providing an aspect ratio of at least 4:1.

### Example 83

The apparatus of any of Examples 76 through 82, the light source comprising a laser diode.

### Example 84

The apparatus of any of Examples 76 through 83, the laser diode comprising a multimode diode, the optical fiber being configured to transmit multiple channels of light.

### Example 85

The apparatus of any of Examples 67 through 84, further comprising a camera system, the camera system being operable to capture images of the reaction sites.

### Example 86

The apparatus of Example 85, the camera system including a time delay integration camera.

### Example 87

The apparatus of any of Examples 85 through 86, the camera system being operable to capture light emitted by fluorophores at the reaction sites in response to the light projected by the illumination assembly,

### Example 88

The apparatus of any of Examples 67 through 87, the illumination assembly further including a refractive substrate, the refractive substrate having a first surface and a second surface, the first lenslet array being disposed on the first surface of the refractive substrate, the second lenslet array being disposed on the second surface of the refractive substrate.

### Example 89

The apparatus of Example 88, refractive substrate being configured such that the first and second surfaces of the refractive substrate are respectively positioned to substantially focus a collimated beam that is incident on the first array onto the second array.

### IV. Miscellaneous

The foregoing teachings may be readily applied in the context of various kinds of camera features in camera system (140). For instance, the foregoing teachings may be applied in the context of a camera system (140) that includes a dual-sensor time delay integration (TDI) camera, a single-sensor camera, a camera with one or more two-dimensional image sensors, and/or other kinds of camera features in camera system (140).

While the foregoing examples are provided in the context of a system (100) that may be used in nucleotide sequencing processes, the teachings herein may also be readily applied in other contexts, including in systems that perform other processes (i.e., other than nucleotide sequencing procedures). The teachings herein are thus not necessarily limited to systems that are used to perform nucleotide sequencing processes.

It is to be understood that the subject matter described herein is not limited in its application to the details of construction and the arrangement of components set forth in the description herein or illustrated in the drawings hereof. The subject matter described herein is capable of other implementations and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

When used in the claims, the term “set” should be understood as one or more things which are grouped together. Similarly, when used in the claims “based on” should be understood as indicating that one thing is determined at least in part by what it is specified as being “based on.” Where one thing is required to be exclusively determined by another thing, then that thing will be referred to as being “exclusively based on” that which it is determined by.

Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings. Also, it is to be understood that phraseology and terminology used herein with reference to device or element orientation (such as, for example, terms like “above,” “below,” “front,” “rear,” “distal,” “proximal,” and the like) are only used to simplify description of one or more examples described herein, and do not alone indicate or imply that the device or element referred to must have a particular orientation. In addition, terms such as “outer” and “inner” are used herein for purposes of description and are not intended to indicate or imply relative importance or significance.

It is to be understood that the above description is intended to be illustrative, and not restrictive. For example, the above-described examples (and/or aspects thereof) may be used in combination with each other. In addition, many modifications may be made to adapt a particular situation or material to the teachings of the presently described subject matter without departing from its scope. While the dimensions, types of materials and coatings described herein are intended to define the parameters of the disclosed subject matter, they are by no means limiting and instead illustrations. Many further examples will be apparent to those of skill in the art upon reviewing the above description. The scope of the disclosed subject matter should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. § 112(f) paragraph, unless and until such claim limitations expressly use the phrase “means for” followed by a statement of function void of further structure.

The following claims recite aspects of certain examples of the disclosed subject matter and are considered to be part of the above disclosure. These aspects may be combined with one another.

1. An apparatus, comprising:
a sequencing stage configured to receive a flow cell comprising a plurality of reaction sites, each reaction site being configured to contain a biological sample; and
an illumination assembly configured to project light toward the sequencing stage to thereby illuminate the reaction sites, the illumination assembly including:
a light source,
a first lenslet array interposed between the light source and the sequencing stage,
a second lenslet array interposed between the light source and the sequencing stage, and
a diffuser interposed between the light source and the sequencing stage.

2. The apparatus of claim 1, the diffuser comprising a one-dimensional diffuser.

3. The apparatus of claim 1, the first lenslet array comprising a cylindrical mircolens array.

4. The apparatus of claim 1, the diffuser being interposed between the light source and the first lenslet array.

5. The apparatus of claim 1, the diffuser being interposed between the first lenslet array and the second lenslet array.

6. The apparatus of claim 1, the second lenslet array being interposed between the first lenslet array and the diffuser.

7. The apparatus of claim 1, further comprising a collimator interposed between the light source and one or both of the first lenslet array or the diffuser.

8. The apparatus of claim 7, the collimator comprising an anamorphic collimator.

9. The apparatus of any claim 1, the first lenslet array comprising a plurality of lenslets, each lenslet having a focal length, the first lenslet array and the second lenslet array being spaced apart from each other by a distance approximately equal to the focal length.

10. The apparatus of claim 1, the light source comprising an optical fiber.

11. The apparatus of claim 10, the optical fiber having a core with a square cross-sectional shape.

12. The apparatus of claim 10, the optical fiber having a core with a circular cross-sectional shape.

13. The apparatus of claim 10, the optical fiber having a core with a rectangular cross-sectional shape defined by a length and a width, the width being greater than the length.

14. The apparatus of claim 13, the length and width providing an aspect ratio of at least 2:1.

15-16. (canceled)

17. The apparatus of claim 10, the light source comprising a laser diode.

18. The apparatus of claim 10, the laser diode comprising a multimode diode, the optical fiber being configured to transmit multiple channels of light.

19. The apparatus of claim 1, further comprising a camera system, the camera system being operable to capture images of the reaction sites.

20. The apparatus of claim 19, the camera system including a time delay integration camera.

21. (canceled)

22. A method comprising:
communicating light through an illumination assembly toward a sequencing stage, the illumination assembly including:
a light source,
a first lenslet array interposed between the light source and the sequencing stage,
a second lenslet array interposed between the light source and the sequencing stage, and
a diffuser interposed between the light source and the sequencing stage;
the communicated light being further communicated toward a plurality of reaction sites at the sequencing stage, each reaction site containing a biological sample.

23-66. (canceled)

67. An apparatus, comprising:
a sequencing stage configured to receive a flow cell comprising a plurality of reaction sites, each reaction site being configured to contain a biological sample; and
an illumination assembly configured to project light toward the sequencing stage to thereby illuminate the reaction sites, the illumination assembly including:
a light source,
a first lenslet array interposed between the light source and the sequencing stage, and
a second lenslet array interposed between the light source and the sequencing stage.

68-89. (canceled)
